(19) United States
(12) Patent Application Publication
(10) Pub. No.: US 2026/0294339 A1
(43) Pub. Date: Oct. 1, 2026

(54) METHOD FOR ANALYZING EATING BEHAVIOR BASED ON BIOLOGICAL SIGNALS

(71) Applicant: PALM FIRM CORP., Seoul (KR)

(72) Inventors: Ho Chun CHOI, Seoul (KR); Min Sung KWON, Seoul (KR)

(73) Assignee: PALM FIRM CORP., Seoul (KR)

(21) Appl. No.: 19/397,068

(22) Filed: Nov. 21, 2025

(30) Foreign Application Priority Data
Feb. 6, 2025 (KR) 10-2025-0015280
Jun. 9, 2025 (KR) 10-2025-0074827

(51) Int. Cl. A61B 5/00; A61B 5/0205; A61B 5/021

(52) U.S. Cl. CPC A61B 5/4866; A61B 5/02055; A61B 5/021; A61B 5/6804; A61B 5/7264; A61B 5/7275

(57) ABSTRACT

A method for analyzing eating behavior based on biological signals is disclosed. The method contains steps of receiving body temperature information, heart rate information, and blood pressure information measured by a body temperature measurement unit, a heart rate measurement unit, and a blood pressure measurement unit provided in a wearable device; generating body temperature derivative information and body temperature integral information; calculating a change rate ratio of the heart rate information and the blood pressure information to generate a cardiovascular response index; inputting the body temperature derivative information, the body temperature integral information, and the cardiovascular response index into a machine learning model to predict eating behavior, meal amount, and/or nutrient composition; and transmitting the prediction result to the user device or linking the prediction result with medical treatment data of a medical institution.

### CROSS-REFERENCE TO RELATED APPLICATIONS

This application claims the benefit of priority under 35 U.S.C. § 119(a) to Korean Patent Application No. 10-2025-0015280, filed on Feb. 6, 2025, and Korean Patent Application No. 10-2025-0074827, filed on Jun. 9, 2025, the entire contents of which are incorporated herein by reference.

### FIELD OF THE INVENTION

The present invention relates to a method and system for analyzing eating behavior based on biological signals, and more particularly, to a method and system for analyzing eating behavior based on biological signals that measure and analyze biological signals through a wearable device worn on or attached to a user's body to predict eating behavior, meal amount, and ingested nutrient composition.

### BACKGROUND ART

Conventional eating behavior monitoring technologies have primarily been implemented by detecting physical behaviors such as wrist movement, chewing sounds, and swallowing motions using accelerometers, gyroscopes, microphones, piezoelectric sensors, and the like. Such methods have limitations in accurate recognition of behavior patterns, and it is difficult to accurately determine whether a user is actually eating.

Existing biological signal measurement and analysis methods have limitations in accurately interpreting various biological signal patterns because they utilize only raw information collected through a single sensor, or the interpretation of collected information is conducted mainly by engineers. In addition, there have been problems of difficulty in discriminating noise/signal patterns with severe individual differences, and high costs and time consumption for post-processing and interpretation.

In particular, research detecting overeating status by utilizing information related to the ratio of heart rate (HR) and blood pressure (BP) or body temperature has not been confirmed to date. Existing studies have only analyzed changes in heart rate, blood pressure, or body temperature after meals individually, and there have been no cases of utilizing them as ratios. Information related to body temperature has also only involved analysis cases using simple measured values, and there have been no cases of precisely analyzing and utilizing the degree of body temperature rise.

### DISCLOSURE OF THE INVENTION

### Problems to be Solved

The present invention is directed to accurately predicting eating behavior, meal amount, nutrient composition, and the like through combined analysis of various biological signals and providing the same to users.

Another object of the present invention is to enable more in-depth analysis by utilizing derivative values and integral values of biological signals, and particularly to detect whether meal amount is excessive or insufficient through a ΔBP/ΔHR ratio.

Another object of the present invention is to enable more in-depth analysis by utilizing derivative values and integral values of biological signals, and particularly to detect whether meal amount is excessive or insufficient through analysis of a rate of baseline body temperature change and an integral value of body temperature change within a preset time range.

A further object of the present invention is to support health management of users through real-time monitoring and AI analysis, and to detect health abnormalities such as postprandial hypotension or diabetic autonomic neuropathy.

A further object of the present invention is to provide automated monitoring services in connection with wearable devices such as smart watches, smart glasses, and smart rings.

A further object of the present invention is to link with diet apps or health management platforms to detect information on overeating or undereating status in real time and provide health feedback.

Objects of the present invention are not limited to those mentioned above, and other objects not mentioned will be clearly understood by those of ordinary skill in the art to which the present invention pertains from the description below.

### SUMMARY OF THE INVENTION

According to one aspect of the present invention for solving the technical problem, a biological signal-based eating behavior analysis method comprises: receiving, by a service server, biological signal data from at least one wearable device worn on or attached to a user's body or from a user device connected to the wearable device; generating processed information from the received biometric information; inputting the generated processed information into a machine learning model to predict eating behavior of the user; and transmitting the predicted eating behavior information to the user device or linking the predicted eating behavior information with medical treatment data of a medical institution.

Specifically, in a method of operating a service server that receives biological signal data from at least one wearable device worn on or attached to a user's body and predicts eating behavior, the method may comprise: receiving, by the service server, body temperature information, heart rate information, and blood pressure information measured by a body temperature measurement unit, a heart rate measurement unit, and a blood pressure measurement unit provided in the wearable device from a user device; generating body temperature derivative information representing a rate of body temperature change per unit time (dT/dt) from the body temperature information, and body temperature integral information representing a time-integrated cumulative value of body temperature elevation relative to a baseline body temperature; calculating a change rate ratio of the heart rate information and the blood pressure information to generate a cardiovascular response index; inputting the body temperature derivative information, the body temperature integral information, and the cardiovascular response index into a machine learning model to predict eating behavior, meal amount, and/or nutrient composition; and transmitting the prediction result to the user device or linking the prediction result with medical treatment data of a medical institution.

Here, the service server may further comprise generating an edge AI unit comprising part or all of the machine learning model, and transmitting the generated edge AI unit to the user device or the wearable device, wherein the edge AI unit may be configured to process and analyze biological signals collected by the wearable device locally in real time and provide immediate feedback without an internet connection.

Here, the edge AI unit transmitted by the service server is optimized for limited computing resources of the wearable device, and may be optimized for hardware performance of the device through quantization and model compression techniques.

Here, the service server may further comprise receiving analysis results processed through the edge AI unit from the wearable device, performing cloud-based secondary analysis based on the analysis results, generating an updated edge AI unit, and retransmitting the updated edge AI unit to the wearable device.

Here, the service server may further comprise periodically updating the machine learning model by learning biometric information and actual eating behavior data collected from a plurality of users.

Here, the service server may comprise generating a user-specific profile of a specific user selected from the plurality of users to build a personalized prediction model, and may further comprise comparing actual eating behavior data and predicted values for an initial period of 5 to 10 days to calibrate model weights in a personalized manner.

Here, the service server may further comprise generating a user-customized edge AI unit based on the user-specific profile and the calibrated model weights and transmitting the user-customized edge AI unit to the user device or the wearable device, thereby enabling personalized eating behavior prediction and analysis to be performed locally.

Here, the cardiovascular response index is defined as a ratio of blood pressure change to heart rate change (ΔBP/ΔHR), and the method may further comprise determining, by the service server, an overeating state when the cardiovascular response index is less than −0.3, generating overeating state information, and transmitting the overeating state information to the user device.

Here, the service server may be configured to determine an overeating state when the cardiovascular response index is less than −0.5, generate overeating state information, and transmit the overeating state information to the user device.

Here, the service server may further comprise estimating calorie intake according to a magnitude of the body temperature integral information, wherein the body temperature integral information correlates with a total amount of diet-induced thermogenesis (DIT), and classifying protein, carbohydrate, and fat ratios according to a pattern of the body temperature derivative information to generate nutrient composition ratio information, and may further comprise transmitting the estimated calorie amount and the nutrient composition ratio information to the user device.

Here, the service server may further comprise generating an edge AI unit comprising an AI model for the calorie estimation and the nutrient composition ratio classification and transmitting the edge AI unit to the wearable device, thereby enabling the wearable device to analyze calorie intake and nutrient composition ratio in real time without an internet connection and provide immediate feedback.

Here, the step of generating the nutrient composition ratio information may be configured to classify as a high-protein diet when a slope of the body temperature derivative information is at a rate of at least 0.3° C. per 20 minutes and not more than 0.7° C. per 20 minutes and a duration is at least 15 minutes and not more than 25 minutes, generate the high-protein diet information, and transmit the high-protein diet information to the user device, or classify as a high-fat diet when the slope of the body temperature derivative information is at a rate of at least 0.1° C. per 20 minutes and not more than 0.3° C. per 20 minutes and the duration is at least 10 minutes and less than 15 minutes, generate high-fat diet information, and transmit the high-fat diet information to the user device.

Here, the step of generating the nutrient composition ratio information may be configured to use a feature set consisting of at least one of slope, change rate, curvature, integral value of the body temperature derivative information, and duration thereof as inputs to a machine learning model, classify a pattern of body temperature derivative information characterized by relatively large slope and long duration observed in high-protein foods (for example, a pattern in which the slope of the body temperature derivative information is maintained at a rate of about 0.3° C. per 20 minutes or more for a certain time) as a high-protein diet, and classify a pattern observed in high-fat foods (for example, a pattern in which the slope of the body temperature derivative information is maintained at a rate of about 0.3° C. per 20 minutes or less for a certain time) as a high-fat diet.

Here, the service server may further comprise determining a measurement error when a correlation coefficient of measured values among a plurality of wearable devices is from 0.5 to less than 0.7, transmitting a self-calibration command to a corresponding device, and receiving a self-calibration result and utilizing the self-calibration result for device performance improvement.

Here, the service server may determine a measurement inconsistency when a correlation coefficient among biological signals of the same type measured by a plurality of wearable devices in the same time period is from 0.5 to less than 0.7 as an example, transmit a self-calibration command to the corresponding device, and the threshold may be adaptively adjusted based on machine learning or statistical criteria according to sensor type, sampling rate, and user characteristics.

Here, the service server may further comprise generating a distributed edge AI unit for self-calibration and data synchronization among the plurality of wearable devices and transmitting the distributed edge AI unit to each wearable device, thereby enabling the plurality of wearable devices to autonomously calibrate and synchronize measured values by communicating with each other.

Here, the service server may further comprise analyzing temporal change patterns of the body temperature information, the heart rate information, and the blood pressure information to distinguish breakfast, lunch, dinner, and snacks; and generating eating habit analysis information based on the distinction result and transmitting the eating habit analysis information to the user device.

Here, the service server may further comprise sharing anonymized biometric information and meal pattern data with a medical research institution with user consent, and may further comprise receiving an improved analysis algorithm from the medical research institution and updating the machine learning model.

Here, the service server may further comprise generating an updated edge AI unit based on the improved analysis algorithm received from the medical research institution and transmitting the updated edge AI unit to the wearable device, thereby enabling analysis reflecting latest medical research results to be performed locally.

According to another aspect of the present invention, there may be provided a service server that communicates with at least one wearable device worn on or attached to a user's body and/or a user device connected to the wearable device to analyze biological signals and predict eating behavior, the service server comprising: a communication unit configured to receive body temperature information, heart rate information, and blood pressure information from the wearable device; a data processing unit configured to generate body temperature derivative information representing a rate of body temperature change per unit time (dT/dt) from the body temperature information, and generate body temperature integral information representing a time-integrated cumulative value of body temperature elevation relative to a baseline body temperature, and generate a cardiovascular response index from the heart rate information and the blood pressure information; an AI analysis unit configured to receive the body temperature derivative information, the body temperature integral information, and the cardiovascular response index as inputs and predict eating behavior, calorie intake, and nutrient composition ratio; a database configured to store data from a plurality of users and maintain user-specific customized models; and a result providing unit configured to transmit prediction results to the user device or link the prediction results with a medical institution server.

Here, the service server may further comprise an edge AI deployment unit configured to generate an edge AI unit comprising part or all of the AI analysis unit and transmit the edge AI unit to the wearable device or the user device, wherein the edge AI unit may be configured to be optimized for hardware performance of the wearable device or the user device and perform real-time local processing and analysis of biological signals.

According to yet another aspect of the present invention for solving the technical problem, a biological signal-based eating behavior analysis method comprises: collecting, by a wearable device worn on or attached to a user's body, biometric information; generating, by an edge AI unit of the wearable device, processed information from the collected biometric information; inputting, by the edge AI unit of the wearable device, the generated processed information into a machine learning model to predict eating behavior, meal amount, and/or nutrient composition of the user; and providing, by the wearable device, the predicted eating behavior information to the user through an output unit or transmitting the predicted eating behavior information to a user device or a service server.

Specifically, in a method in which a wearable device worn on or attached to a user's body collects biometric information and an edge AI unit included in the wearable device analyzes the collected biometric information to predict eating behavior, the method may comprise: collecting, by the wearable device, body temperature information, heart rate information, and blood pressure information through a body temperature measurement unit, a heart rate measurement unit, and a blood pressure measurement unit, respectively; generating, by the edge AI unit of the wearable device, body temperature derivative information representing a rate of body temperature change per unit time (dT/dt) from the body temperature information, and body temperature integral information representing a time-integrated cumulative value of body temperature elevation relative to a baseline body temperature; and calculating, by the edge AI unit of the wearable device, a change rate ratio of the heart rate information and the blood pressure information to generate a cardiovascular response index.

Here, the wearable device may be configured as two or more independent devices selected from a wrist-worn device, an ear-worn device, a chest-attached device, and a glasses-type device, and may further comprise performing, by the wearable device, self-calibration when a correlation coefficient of measured values among the devices is from 0.5 to less than 0.7.

Here, the body temperature integral information is a cumulative value of body temperature rise due to diet-induced thermogenesis (DIT), and may be generated through: continuously measuring, by the wearable device, body temperature of the user; calculating, by the edge AI unit of the wearable device, a body temperature change rate over time from the measured body temperature data; and determining, by the edge AI unit of the wearable device, rapid food intake when the calculated body temperature change rate exceeds a predetermined threshold.

Here, the edge AI unit of the wearable device may be configured to generate and output rapid eating state information when a section in which the body temperature change rate is at a rate of 0.3° C. per 10 minutes or more and continues for 10 minutes or more is confirmed within a measurement period of 30 to 40 minutes.

Here, the cardiovascular response index is defined as a ratio of blood pressure change to heart rate change (ΔBP/ΔHR), and the method may further comprise determining, by the edge AI unit of the wearable device, an overeating state when the cardiovascular response index is less than −0.3, generating overeating state information, and outputting the overeating state information.

Here, the edge AI unit of the wearable device may be configured to determine an overeating state when the cardiovascular response index is less than −0.5, generate overeating state information, and output the overeating state information.

Here, the method may further comprise a preprocessing step in which the edge AI unit of the wearable device distinguishes exercise-induced body temperature rise from meal-induced body temperature rise using exercise information collected through an accelerometer provided in the wearable device.

Here, the method may further comprise: inputting, by the edge AI unit of the wearable device, the body temperature derivative information, the body temperature integral information, and the cardiovascular response index into a machine learning model to predict eating behavior, meal amount, and/or nutrient composition; and transmitting, by the wearable device, the prediction result to a user device or a service server to provide feedback, wherein the machine learning model is configured with a hybrid structure of Long Short-Term Memory (LSTM) and Temporal Convolutional Network (TCN), and may be configured to simultaneously analyze a standard deviation of the body temperature derivative information, a coefficient of variation of the cardiovascular response index, and a slope of the body temperature integral information within a time window of 10 to 20 minutes.

Here, the method may further comprise estimating, by the edge AI unit of the wearable device, calorie intake according to a magnitude of the body temperature integral information, wherein the body temperature integral information correlates with a total amount of diet-induced thermogenesis (DIT), and classifying protein, carbohydrate, and fat ratios according to a pattern of the body temperature derivative information.

Here, the edge AI unit of the wearable device may be configured to classify as a high-protein diet when a slope of the body temperature derivative information is at a rate of at least 0.3° C. per 20 minutes and not more than 0.7° C. per 20 minutes and a duration is at least 15 minutes and not more than 25 minutes, generate high-protein diet information, and transmit the high-protein diet information to the user device or the service server, and classify as a high-fat diet when the slope of the body temperature derivative information is at a rate of at least 0.1° C. per 20 minutes and not more than 0.3° C. per 20 minutes and the duration is at least 10 minutes and less than 15 minutes, generate high-fat diet information, and transmit the high-fat diet information to the user device or the service server.

Here, the method may further comprise receiving and installing, by the wearable device, an edge AI unit from a service server or a user device, wherein the received edge AI unit may be configured to include an AI model optimized for hardware performance of the wearable device.

Here, the method may further comprise transmitting, by the wearable device, eating behavior prediction results, meal amount estimation results, and nutrient composition ratio analysis results generated by the edge AI unit, together with body temperature information, heart rate information, and blood pressure information measured at regular intervals over a 24-hour cycle, to the service server, and receiving an updated edge AI unit with higher prediction accuracy than a previous version from the service server and automatically updating the edge AI unit.

### Advantageous Effects

The method for analyzing eating behavior based on biological signals according to the present invention can accurately predict eating behavior, meal amount, nutrient composition, and the like through combined analysis of various biological signals and provide the same to users.

The method for analyzing eating behavior based on biological signals according to the present invention can enable more in-depth analysis by utilizing derivative values and integral values of biological signals, and can detect whether meal amount is excessive or insufficient, particularly through a ΔBP/ΔHR ratio.

The method for analyzing eating behavior based on biological signals according to the present invention can enable more in-depth analysis by utilizing derivative values and integral values of biological signals, and can detect whether meal amount is excessive or insufficient through analysis of a rate of baseline body temperature change and an integral value of body temperature change within a preset time range.

The method for analyzing eating behavior based on biological signals according to the present invention can support health management of users through real-time monitoring and AI analysis, and can detect health abnormalities such as postprandial hypotension or diabetic autonomic neuropathy.

The method for analyzing eating behavior based on biological signals according to the present invention can provide automated monitoring services in connection with wearable devices such as smart watches, smart glasses, and smart rings.

The method for analyzing eating behavior based on biological signals according to the present invention can link with diet apps or health management platforms to detect eating behavior information regarding whether meal amount is excessive or insufficient in real time and provide health feedback.

Effects of the present invention are not limited to those mentioned above, and other effects not mentioned will be clearly understood by those of ordinary skill in the art to which the present invention pertains from the description below.

### BRIEF DESCRIPTION OF THE DRAWINGS

FIG. 1 is a flowchart of a method for analyzing eating behavior based on biological signals according to the present invention.

FIG. 2 is a flowchart of a method for analyzing eating behavior based on biological signals according to another embodiment of the present invention.

FIG. 3 is a system configuration diagram according to the present invention.

### DETAILED DESCRIPTION OF THE INVENTION

Objects and effects of the present invention, and technical configurations for achieving them will become clear with reference to the embodiments described in detail below together with the accompanying drawings. In describing the present invention, if it is determined that a detailed description of a known function or configuration may unnecessarily obscure the gist of the present invention, the detailed description thereof will be omitted. Furthermore, the terms described below are terms defined in consideration of functions in the present invention, and may vary depending on the intention or custom of a user or operator.

However, the present invention is not limited to the embodiments disclosed below, but may be implemented in various different forms. The present embodiments are provided only to make the disclosure of the present invention complete, and to fully inform those of ordinary skill in the art to which the present invention pertains of the scope of the invention, and the present invention is only defined by the scope of the claims. Therefore, the definition should be made based on the content throughout the specification.

The main terms used in this specification are defined as follows.

“Body temperature derivative information” means a rate of body temperature change per unit time (dT/dt), where T is the measured body temperature and t is time. For example, “0.3° C. per 20 minutes” indicates that the body temperature change measured over 20 minutes averages 0.3° C. In addition, the machine learning model adaptively calculates the slope according to user-specific baseline information and window size.

“Body temperature integral information” means a time-integrated value (∫(T−T₀)dt) of the cumulative body temperature rise relative to a baseline body temperature (T₀) during a measurement period, where T is the measured body temperature, T₀ is the baseline body temperature, and t is time. This is a cumulative thermal energy indicator correlated with diet-induced thermogenesis (DIT), and can be utilized as a feature based on continuous interval area rather than an absolute value.

“Pattern of body temperature derivative information” means a trend of increase, decrease, or maintenance of the body temperature change rate over time. That is, it means a temporal change pattern of the slope at which body temperature rises. Specifically, the pattern of body temperature derivative information may mean a temporal change aspect consisting of a combination of one or more derived features such as slope (trend of increase, decrease, or maintenance of body temperature over time), change rate, curvature, amplitude, duration, and distribution of change rate.

“Duration of body temperature derivative information” means the time during which the body temperature change rate remains within a specific range, that is, the time during which body temperature elevation above baseline is sustained. Specifically, the duration of body temperature derivative information can be understood as representing a time interval during which a specific feature is continuously maintained above or below a threshold. For example, “at least 15 minutes and not more than 25 minutes” indicates that the body temperature change rate is maintained within the specified range for a period ranging from 15 to 25 minutes. In addition, the threshold and time measurement criteria may be configured such that the machine learning model learns user-specific physiological variability and dynamically adjusts them.

“Cardiovascular response index (CRI, Cardiovascular Response Index)” is an indicator representing the ratio of changes in blood pressure (BP) and heart rate (HR), which can be calculated as ΔBP/ΔHR or ΔHR/ΔBP. Alternatively, it may mean one or more features including ΔBP, ΔHR, and their ratios. Unless otherwise specified, the examples in this specification use the ΔBP/ΔHR method, where Δ means a change amount. In addition, the negative threshold or positive threshold presented in this specification is an exemplary criterion of one embodiment, and the machine learning model may be configured to automatically calculate personalized judgment criteria by reflecting user-specific baseline information, age, autonomic nervous system characteristics, and the like.

Throughout the specification, when a part is said to “comprise” or “include” a component, this means that it may further include other components, rather than excluding other components, unless specifically stated to the contrary. In addition, terms such as “ . . . unit”, “ . . . part”, or “ . . . module” described in the specification mean a unit that processes at least one function or operation, which may be implemented by hardware or software, or a combination of hardware and software.

Meanwhile, in the embodiment of the present invention, each component, functional block, or means may be composed of one or more sub-components, and electrical, electronic, and mechanical functions performed by each component may be implemented by various known elements such as electronic circuits, integrated circuits, ASICs (Application Specific Integrated Circuits), or mechanical elements, and may be implemented separately or two or more may be integrated into one.

In addition, combinations of each block in the attached block diagram and each step in the flowchart may be performed by computer program instructions. These computer program instructions may be mounted on a processor of a general-purpose computer, special-purpose computer, portable notebook computer, network computer, mobile device such as a smartphone, online service providing server, or other programmable data processing equipment, so that the instructions executed through the processor of the computer device or other programmable data processing equipment create means for performing the functions described in each block of the block diagram or each step of the flowchart described below. These computer program instructions may also be stored in computer-usable memory or computer-readable memory that can direct a computer device or other programmable data processing equipment to implement functions in a specific manner, so it is also possible to produce an article of manufacture containing instruction means for performing the functions described in each block of the block diagram or each step of the flowchart. Since computer program instructions can also be mounted on a computer device or other programmable data processing equipment, it is also possible to provide steps for executing the functions described in each block of the block diagram and each step of the flowchart by creating a process for performing a series of operational steps on the computer device or other programmable data processing equipment.

In addition, each block or each step may represent a module, segment, or part of code including one or more executable instructions for executing specified logical function(s). It should also be noted that in some alternative embodiments, the functions mentioned in the blocks or steps may occur out of order. For example, two blocks or steps shown in succession may in fact be performed substantially simultaneously, or the blocks or steps may sometimes be performed in reverse order depending on the corresponding function.

In the embodiment of the present invention, a user device or wearable device refers to any computing means capable of collecting, reading, processing, working, storing, and displaying data, such as desktop computers, notebook computers, smartphones, PDAs, mobile phones, game consoles, smart watches, smart glasses, smart rings, and the like. In particular, the user device in the embodiment of the present invention is a device having a function capable of executing software written in decodable code and displaying and delivering the same to the user. In addition, if necessary, the software may be stored by itself, or may be read together with data from the outside. In addition, when the user device and the wearable device are “connected”, it may mean a state in which data is transmitted and received in real time between the two devices through wireless or wired communication, and mutual information exchange and control are possible. For example, this corresponds to a case where a smartphone and a smart watch or smart glasses are connected wirelessly such as via Bluetooth to exchange information such as text messages, phone calls, health data, and the like, and interact with each other according to user commands. That is, “connected” may mean not simply being physically close, but being in a communication state in which the two devices recognize each other and can synchronize data or exchange commands.

In addition, the terminal in the embodiment of the present invention includes functions such as input, output, and storage as well as the above data processing functions, and for this purpose, various elements such as CPU, motherboard, graphics card, hard disk, sound card, speakers, keyboard, mouse, monitor, USB, communication modem, etc. that general computer devices have, as well as CPU, motherboard, graphics chip, memory chip, sound engine, speakers, touch pad, external connection terminals such as USB, communication antenna, communication modems capable of implementing communication such as 3G, LTE, LTE-A, WiFi, Bluetooth, etc. that wireless smartphone terminals have, may be included. These various elements may be used alone or two or more together, or parts of various elements may be combined to implement one or more functions, and in the embodiment of the present invention, a device or parts thereof indicated by one or more blocks in the drawings or detailed description may mean that various elements included in the above user device are used alone or two or more together, or parts of various elements are combined to represent one or more functions.

“Edge AI (Edge Artificial Intelligence)” used in the present invention means a technology that operates an artificial intelligence model on a device (edge device) where data is generated, rather than in a central server or cloud environment, to collect, process, and analyze data. Specifically, it refers to an artificial intelligence technology that directly collects biological signals from edge devices such as wearable devices or user devices and performs real-time analysis and prediction based thereon.

In the present invention, an “edge AI unit” may be defined as a software module including part or all of a machine learning model generated in a service server and transmitted to a user device or wearable device. The edge AI unit is configured to process and analyze biological signals collected by the wearable device locally in real time and provide immediate feedback without an internet connection. The “edge AI unit” is optimized for limited computing resources of the wearable device, and may be provided optimized for hardware performance of the device through model compression techniques such as quantization, pruning, knowledge distillation, and the like.

In addition, the “edge AI unit” is configured in a user-customized manner based on a user-specific profile and calibrated model weights, and is configured to enable personalized eating behavior prediction and analysis to be performed locally.

The edge AI included in the “edge AI unit” of the present invention may be designed to enable efficient computation even in a limited hardware environment by including a lightweight artificial intelligence model for calorie estimation and nutrient composition ratio classification. A distributed edge AI structure for self-calibration and data synchronization among a plurality of wearable devices may be adopted so that devices can autonomously calibrate and synchronize measured values by communicating with each other. A hybrid learning structure may be retained in which analysis results from local processing are transmitted to a service server, the server performs cloud-based secondary analysis based thereon, generates an edge AI unit with updated parameters or weights, and retransmits it to the device.

The edge AI unit of the wearable device can directly generate body temperature derivative information, which is a time-derivative value from body temperature information, and body temperature integral information, which is an integral value relative to a baseline body temperature. In addition, the edge AI unit can generate a cardiovascular response index by calculating a change rate ratio of heart rate information and blood pressure information in real time. Based on the generated information, eating behavior, meal amount, and nutrient composition can be immediately predicted locally and provided to the user, and the wearable device or the user device connected to the wearable device transmits biometric information measured at regular intervals together with analysis results generated by the edge AI unit to the service server, receives an updated edge AI unit with improved accuracy from the server, and automatically updates it.

The service server can generate an updated edge AI unit based on an improved analysis algorithm received from a medical research institution so that the latest medical research results can be reflected in local analysis. The edge AI defined as such is used in the method for analyzing eating behavior based on biological signals of the present invention, and can be understood as a means for efficiently providing real-time analysis and feedback for user health management and eating habit improvement.

Meanwhile, in the embodiment of the present invention, user devices, wearable devices, and the like may have communication functions, and may be equipped with various network means such as wired internet, wireless internet, infrared communication, Bluetooth, WCDMA, Wibro, WiFi, LTE, LTE-A, 5G wired and wireless telephone networks, and the like to implement communication functions.

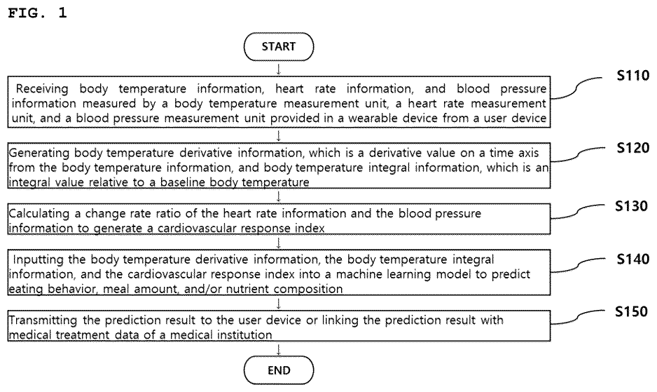

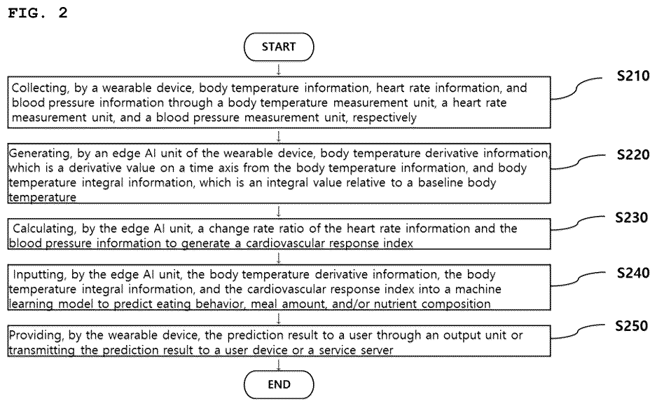

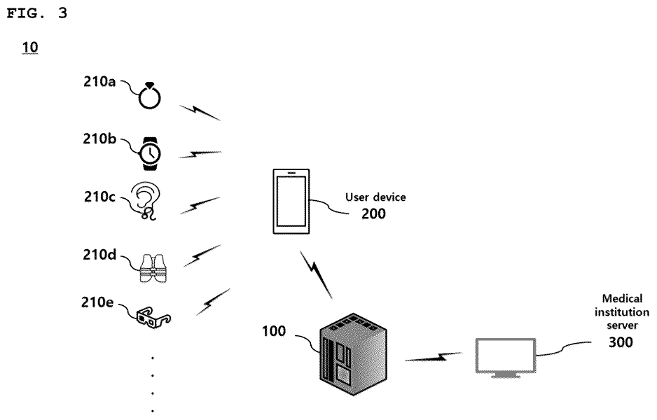

FIG. 1 is a flowchart of a method for analyzing eating behavior based on biological signals according to the present invention, FIG. 2 is a flowchart of a method for analyzing eating behavior based on biological signals according to another embodiment of the present invention, and FIG. 3 is a system configuration diagram according to the present invention. Hereinafter, the method for analyzing eating behavior based on biological signals according to an embodiment of the present invention will be described in detail.

In describing the present invention, biometric information means information measured from a user's body, such as heart rate (HR), blood pressure (BP), body temperature (BT), respiration rate (RR), and oxygen saturation (SpO2).

In describing the present invention, eating behavior can be understood to include at least one or more of information on meal start, duration, and meal end.

In describing the present invention, processed information is information generated by differentiating, integrating, or combining collected biometric information, and includes body temperature derivative information, body temperature integral information, cardiovascular response index, and the like.

Body temperature derivative information is information generated by differentiating body temperature information with respect to time, and represents the rate of body temperature rise.

Body temperature integral information is information generated by integrating the excess of body temperature information over baseline body temperature with respect to time, and represents the cumulative value of elevated body temperature.

Cardiovascular response index is an index representing the ratio of heart rate change information and blood pressure change information, and is used to determine whether overeating has occurred.

Diet-Induced Thermogenesis (DIT) means body temperature rise occurring during the digestion process after food intake.

The present invention relates to a service server (100) and a for predicting a user's eating behavior and analyzing meal amount and nutrient composition by analyzing biological signals collected from a wearable device (210), wherein the service server (100) of the present invention comprises a communication unit, a data processing unit, an AI analysis unit, a database, a result providing unit, and an edge AI deployment unit.

In the description of the present invention, the definition and calculation method of the cardiovascular response index (CRI) are used as a basis. The cardiovascular response index is defined as the ratio (ΔBP/ΔHR) of blood pressure change (ΔBP) and heart rate change (ΔHR), and is used as a key indicator for quantifying hemodynamic load due to meals.

For example, in a normal meal situation, heart rate increases in the range of 4-10 bpm and blood pressure decreases by 0-5 mmHg, which means that increased blood flow to the digestive organs is balanced by the cardiovascular system. On the other hand, during overeating, the CRI value appears below −0.3, which indicates that peripheral blood flow reduction due to splanchnic pooling has occurred and the compensatory mechanism of the autonomic nervous system has reached its limit. Through fusion analysis with body temperature derivative and integral information, body temperature derivative information (dT/dt) suggests a high-protein diet when a rapid rise pattern of 0.3-0.7° C. is observed 5-15 minutes after the start of a meal, and body temperature integral information (∫(T−T₀)dt) indicates a possibility of overeating when a cumulative rise of 0.5° C. or more is observed over 30-40 minutes. In particular, when the CRI value is less than −0.3 and the body temperature integral information is 15-20° C.·min or more, it is comprehensively determined as a high-calorie overeating state. For personalized calibration of the machine learning model, the LSTM-Transformer hybrid model simultaneously analyzes the standard deviation of body temperature derivative information, the coefficient of variation of CRI, and the slope of body temperature integral information within a time window of 10-20 minutes, and performs personalized model weight calibration reflecting individual metabolic differences using user data for an initial period of 5-10 days.

In the embodiment of the present invention, when the CRI value is less than −0.3, overeating status can be detected in real time with 89% accuracy, postprandial hypotension with blood pressure drop of 20 mmHg or more can be automatically detected, and signs of autonomic nervous system abnormalities appearing as CRI values of +0.3 or more in diabetic patients can be detected early. In addition, when the slope of body temperature derivative information is 0.3-0.7° C. per 20 minutes and the CRI value is −0.3 to −0.5, high-protein diet is classified with 82% accuracy, and when the slope of body temperature derivative information is less than 0.3° C. per 20 minutes and the CRI value is less than −0.3, high-fat diet is identified with 75% accuracy, thereby greatly improving nutrient composition classification performance.

Hereinafter, each component of the service server (100) and its functions will be described in detail.

First, the communication unit is configured to receive body temperature information, heart rate information, and blood pressure information from at least one wearable device (210) worn on or attached to the user's body and/or from a user device (200) connected to the wearable device (210). The communication unit supports various communication protocols (Bluetooth, WiFi, NFC, 5G, etc.) to ensure compatibility with various wearable devices (210). In addition, the communication unit includes error detection and correction algorithms to minimize packet loss that may occur during data transmission. The communication unit includes detailed modules such as a protocol conversion module, a data compression/decompression module, a secure communication module, and a connection management module. The protocol conversion module converts communication protocols of various wearable devices (210) into an internal data format of the service server (100), and the data compression/decompression module is responsible for data compression and decompression processing for bandwidth efficiency. The secure communication module is configured to perform encryption and authentication processing for secure transmission of biometric information, and the connection management module is responsible for simultaneous connection with multiple devices and session management. The communication unit may also receive metadata such as battery status and measurement accuracy of the wearable device (210), which can be used to evaluate data reliability.

The data processing unit is configured to generate body temperature derivative information, body temperature integral information, and cardiovascular response index based on body temperature information, heart rate information, and blood pressure information received from the communication unit. The body temperature derivative information generation module generates an indicator representing the rate of body temperature change over time, and applies moving average filtering for noise removal. This module calculates a differential value using the central difference method and performs statistical filtering for outlier removal. Body temperature derivative information is used as an important indicator for detecting body temperature changes at the time of meal start. The body temperature integral information generation module generates an indicator representing the integral value of body temperature relative to baseline body temperature, sets the baseline body temperature, calculates cumulative integration, and performs time window-based integral value normalization. Body temperature integral information is used to measure the cumulative effect of body temperature rise due to meals. The cardiovascular response index generation module generates an indicator defined as the ratio of blood pressure change and heart rate change, calculates blood pressure change and heart rate change relative to the baseline state, calculates this ratio, applies time weighting, and normalizes it. The cardiovascular response index is used as an important indicator for determining meal amount and whether overeating has occurred. The data processing unit also includes preprocessing functions such as missing value interpolation, time synchronization, and feature extraction.

The AI analysis unit receives body temperature derivative information, body temperature integral information, and cardiovascular response index generated by the data processing unit and performs the role of predicting eating behavior, calorie intake, and nutrient composition ratio. The eating behavior prediction module performs LSTM network-based time series analysis, detects rapid change patterns in body temperature derivative information, recognizes specific patterns of cardiovascular response index, and learns and applies user-specific eating patterns. The calorie intake estimation module analyzes the correlation between the magnitude of body temperature integral information and calorie intake, models the relationship between change patterns of cardiovascular response index and meal amount, and performs calibration considering the user's physical characteristics. The nutrient composition ratio analysis module classifies protein, carbohydrate, and fat ratios according to patterns of body temperature derivative information, analyzes temporal change patterns of cardiovascular response index, and utilizes a classification algorithm based on body temperature change characteristics for each nutrient. The AI analysis unit enhances prediction accuracy by utilizing various machine learning algorithms and improves models through continuous learning. It may also be configured to perform uncertainty quantification, explainable AI, and abnormal pattern detection.

The database is responsible for storing data from multiple users and maintaining user-specific customized models. The database stores and manages information such as user profile information, raw biological signal data, processed feature data, user-specific customized model parameters, meal record data, and feedback data. The database provides functions such as data encryption, data backup and recovery, data version management, and distributed storage.

The result providing unit is responsible for transmitting prediction results of the AI analysis unit to the user device (200) or linking with a medical institution server (300). The user interface generation module is responsible for visualization of prediction results, generation of customized feedback, and generation of notifications. The medical institution linkage module is responsible for conversion to medical standard format, secure communication with the medical institution server (300), and provision of detailed analysis data for medical professionals. The result providing unit also includes functions such as support for various platforms, real-time notifications, and periodic reports.

The edge AI deployment unit is responsible for generating an edge AI unit including part or all of the AI analysis unit and transmitting it to the wearable device (210) or user device (200). The model lightweighting module reduces model size through model compression, quantization, and knowledge distillation, increases computational efficiency, and transfers knowledge from complex models to simple models. The device optimization module performs hardware profiling, resource allocation optimization, and power efficiency optimization to provide optimal performance for target devices. The deployment management module is responsible for version management, remote updates, and performance monitoring. The edge AI unit may be configured to enable latency reduction, enhanced privacy protection, reduced network dependency, and server load distribution by performing real-time local processing and analysis of biological signals on the wearable device (210) or user device (200).

The service server (100) forms a workflow in which the communication unit receives biological signal data from the wearable device (210), the data processing unit preprocesses the received data and extracts features, the AI analysis unit predicts meal-related information based on the extracted features, the database stores raw data, processing results, and prediction results, the result providing unit transmits prediction results to the user device (200) or links with the medical institution server (300), and the edge AI deployment unit deploys the optimized model to the wearable device (210) or user device (200). The service server (100) has operational characteristics such as scalability, availability, security, and maintainability. The service server (100) of the present invention provides personalized health management services by analyzing biological signals collected from the wearable device (210) to predict the user's eating behavior, meal amount, and nutrient composition, and in particular, by introducing a distributed processing method through the edge AI unit, real-time analysis and feedback are possible, and the quality of health management can be improved through linkage with medical institutions. Hereinafter, the method for analyzing eating behavior based on biological signals through the service server (100) will be described in detail.

The method for analyzing eating behavior based on biological signals through the service server (100) according to an embodiment of the present invention is for predicting a user's eating behavior and analyzing meal amount and nutrient composition by analyzing biological signals collected through a wearable device (210), and can provide personalized health management services by analyzing eating patterns using biological signals such as the user's body temperature, heart rate, and blood pressure.

The method for analyzing eating behavior based on biological signals of the present invention relates to an operation method of a service server (100) that predicts a user's eating behavior, meal amount, and nutrient composition based on biological signal data collected from a wearable device (210), and each step will be described in detail below.

The method for analyzing eating behavior based on biological signals according to an embodiment of the present invention includes receiving body temperature information, heart rate information, and blood pressure information measured by a body temperature measurement unit, a heart rate measurement unit, and a blood pressure measurement unit provided in the wearable device (210) from a user device (200) (S110).

In step (S110), the service server (100) receives body temperature information, heart rate information, and blood pressure information measured by a body temperature measurement unit, a heart rate measurement unit, and a blood pressure measurement unit provided in the wearable device (210) from a user device (200).

The wearable device (210) is in a form worn on or attached to the user's body, and the body temperature measurement unit measures the user's body temperature in real time, the heart rate measurement unit measures the user's heart rate, and the blood pressure measurement unit measures the user's blood pressure. These measurements may be performed continuously or at set time intervals.

The service server (100) receives biological signal data from the wearable device (210) through the user device (200) (e.g., smartphone, tablet, etc.), and in this process, various communication protocols such as Bluetooth, WiFi, NFC, etc. may be utilized.

In the receiving step (S110), biological signals of the same type may be received from a plurality of wearable devices (210), and in this case, the service server (100) may calculate a correlation coefficient of measured values among the plurality of wearable devices (210) to determine whether a measurement error exists. If the correlation coefficient is from 0.5 to less than 0.7, it may be determined as a measurement error and a self-calibration command may be transmitted to the corresponding device.

Next, it includes generating body temperature derivative information, which is a derivative value on a time axis from the body temperature information, and body temperature integral information, which is an integral value relative to a baseline body temperature (S120).

In step (S120), the service server (100) generates body temperature derivative information, which is a derivative value on a time axis from the body temperature information, and body temperature integral information, which is an integral value relative to a baseline body temperature.

The body temperature derivative information is an indicator representing the rate of body temperature change over time, and is used for the purpose of detecting body temperature changes at the time of meal start. Body temperature derivative information can be calculated as follows:

$$Body temperature derivative information = dT / dt$$

where T represents body temperature and t represents time.

The body temperature integral information is an indicator representing the integral value of body temperature relative to baseline body temperature, and is used to measure the cumulative effect of body temperature rise due to meals. Body temperature integral information can be calculated as follows:

$$Body temperature integral information = \int ( T - {T}_{0} ) dt$$

where T represents body temperature, T₀ represents baseline body temperature, and t represents time.

The pattern of body temperature derivative information shows different characteristics depending on the nutrient composition ingested, and in particular, the slope and duration of body temperature derivative information become important indicators for classifying the nutrient composition ratio of protein, carbohydrate, fat, etc. For example, when the slope of body temperature derivative information is at a rate of at least 0.3° C. per 20 minutes and not more than 0.7° C. per 20 minutes and the duration is at least 15 minutes and not more than 25 minutes, it can be classified as a high-protein diet.

Next, it includes calculating a change rate ratio of the heart rate information and blood pressure information to generate a cardiovascular response index (S130).

In step (S130), the service server (100) calculates a change rate ratio of heart rate information and blood pressure information to generate a cardiovascular response index.

The cardiovascular response index is defined as the ratio of blood pressure change to heart rate change (ΔBP/ΔHR), and is an indicator that quantifies the cardiovascular system's response to meals. This index is calculated as follows:

$$Cardiovascular response index = Δ BP / Δ HR$$

where ΔBP represents the change in blood pressure and ΔHR represents the change in heart rate.

The cardiovascular response index is used as an important indicator for determining meal amount and whether overeating has occurred. In particular, when the cardiovascular response index is less than −0.3, it is determined as an overeating state, and when it is less than −0.5, it can be determined as a severe overeating state. In such cases, the service server (100) may generate overeating state information and transmit it to the user device (200).

The cardiovascular response index can also be used for long-term eating habit pattern analysis, and can be used to distinguish breakfast, lunch, dinner, snacks, etc. through change patterns over time.

Next, it includes inputting the body temperature derivative information, body temperature integral information, and cardiovascular response index into a machine learning model to predict eating behavior, meal amount, and/or nutrient composition (S140).

In step (S140), the service server (100) inputs body temperature derivative information, body temperature integral information, and cardiovascular response index into a machine learning model to predict eating behavior, meal amount, and/or nutrient composition.

The machine learning model is built by learning biometric information and actual eating behavior data (actual event data recorded by users or obtained based on sensors/logs) collected from a plurality of users, and the service server (100) periodically updates it. Such updates are made in a direction to improve the accuracy of the model through comparison between actual data and eating behavior prediction values estimated from biological signals.

The machine learning model can be built in a personalized manner to reflect user-specific characteristics. The service server (100) may generate a user-specific profile of a specific user selected from a plurality of users, and may calibrate model weights in a personalized manner by comparing actual eating behavior data and predicted values for an initial period of 5 to 10 days.

The machine learning model may estimate calorie intake according to the magnitude of body temperature integral information, and generate nutrient composition ratio information by classifying protein, carbohydrate, and fat ratios according to the pattern of body temperature derivative information. Such analysis helps improve the user's eating habits by providing information on the qualitative aspect of meals.

Next, it includes transmitting the prediction result to the user device (200) or linking with medical treatment data of a medical institution (S150).

In step (S150), the service server (100) transmits the prediction result to the user device (200) or links with medical treatment data of a medical institution.

The prediction result includes eating behavior, meal amount, nutrient composition ratio, etc., and may be visually provided to the user through an application of the user device (200). Such information can be used for improving the user's eating habits and health management.

The linkage with medical treatment data of a medical institution is performed with the user's consent, and through this, medical professionals can comprehensively analyze the user's eating habits and related health information. In addition, the service server (100) may share anonymized biometric information and meal pattern data with medical research institutions with user consent, receive improved analysis algorithms from the institutions, and update the machine learning model.

The result providing step may additionally include analyzing temporal change patterns of body temperature information, heart rate information, and blood pressure information to distinguish breakfast, lunch, dinner, and snacks, and generating eating habit analysis information based thereon and transmitting it to the user device (200).

The present invention includes not only a cloud-based service server (100), but also an edge AI unit that performs local processing on a wearable device (210) or user device (200).

The service server (100) may generate an edge AI unit including part or all of the machine learning model, and transmit it to the user device (200) or wearable device (210). The edge AI unit can process and analyze biological signals collected by the wearable device (210) locally in real time and provide immediate feedback without an internet connection.

The edge AI unit is implemented to be optimized for limited computing resources of the wearable device (210), and is optimized for hardware performance of the wearable device (210) through quantization and model compression techniques. This enables efficient AI computation even on the wearable device (210).

The service server (100) may receive analysis results processed through the edge AI unit from the wearable device (210), perform cloud-based secondary analysis based thereon, generate an updated edge AI unit, and retransmit it to the wearable device (210). This distributed processing method enables utilization of the advantages of both real-time processing and advanced analysis.

By generating a user-customized edge AI unit based on user-specific profile and calibrated model weights and transmitting it to the user device (200) or wearable device (210), personalized eating behavior prediction and analysis can be performed locally.

The method for analyzing eating behavior based on biological signals according to an embodiment of the present invention can provide personalized health management services by analyzing biological signals collected from the wearable device (210) to predict the user's eating behavior, meal amount, and nutrient composition. In particular, it has a technical feature of analyzing eating patterns using body temperature derivative information, body temperature integral information, and cardiovascular response index calculated from biological signals such as body temperature, heart rate, and blood pressure.

In addition, by introducing a distributed processing method through the edge AI unit, real-time analysis and feedback are possible without an internet connection, and the quality of health management can be improved through linkage with medical institutions. Through this, it can contribute not only to personal health management but also to the medical research field, and can enhance the utilization value of wearable devices (210).

According to another embodiment of the present invention, it is for predicting a user's eating behavior and analyzing meal amount and nutrient composition by analyzing biological signals collected through a wearable device (210), and can provide personalized health management services by analyzing eating patterns using biological signals such as the user's body temperature, heart rate, and blood pressure.

The method for analyzing eating behavior based on biological signals according to another embodiment of the present invention relates to a method in which a wearable device (210) collects biometric information and an edge AI unit analyzes the collected information to predict eating behavior, and each step will be described in detail below.

First, it includes the wearable device (210) collecting body temperature information, heart rate information, and blood pressure information through a body temperature measurement unit, a heart rate measurement unit, and a blood pressure measurement unit, respectively (S210).

In step (S210), the wearable device (210) collects body temperature information, heart rate information, and blood pressure information through a body temperature measurement unit, a heart rate measurement unit, and a blood pressure measurement unit, respectively.

The wearable device (210) is worn on or attached to the user's body in the form of a finger-worn device (210a), a wrist-worn device (210b), an ear-worn device (210c), a chest-attached device (210d), a glasses-type device (210e), etc., and measures biological signals. These measurements may be performed continuously or at set time intervals.

The wearable device (210) may calculate a correlation coefficient of measured values among a plurality of devices to determine whether a measurement error exists. If the correlation coefficient is from 0.5 to less than 0.7, it may be determined as a measurement error and self-calibration may be performed.

In addition, by utilizing exercise information collected through an accelerometer provided in the wearable device (210), the edge AI unit may perform a preprocessing process to distinguish exercise-induced body temperature rise from meal-induced body temperature rise.

Next, it includes the edge AI unit of the wearable device (210) generating body temperature derivative information, which is a derivative value on a time axis from the body temperature information, and body temperature integral information, which is an integral value relative to a baseline body temperature (S220).

In step (S220), the edge AI unit of the wearable device (210) generates body temperature derivative information, which is a derivative value on a time axis from the body temperature information, and body temperature integral information, which is an integral value relative to a baseline body temperature.

The body temperature derivative information is an indicator representing the rate of body temperature change over time, and is used for the purpose of detecting body temperature changes at the time of meal start. Body temperature derivative information can be calculated as follows:

$$Body temperature derivative information = dT / dt$$

where T represents body temperature and t represents time.

The body temperature integral information is an indicator representing the integral value of body temperature relative to baseline body temperature, and is used to measure the cumulative effect of body temperature rise due to meals. Body temperature integral information can be calculated as follows:

$$Body temperature integral information = \int ( T - {T}_{0} ) dt$$

where T represents body temperature, T₀ represents baseline body temperature, and t represents time.

The body temperature integral information is a cumulative value of body temperature rise due to diet-induced thermogenesis, and the wearable device (210) continuously measures the user's body temperature, and the edge AI unit calculates the body temperature change rate over time from the measured body temperature data. When the calculated body temperature change rate exceeds a predetermined threshold, it can be determined as rapid food intake.

In particular, when a section in which the body temperature rise rate is at a rate of 0.3° C. per 10 minutes or more and continues for 10 minutes or more is confirmed within a measurement period of 30-40 minutes, rapid eating state information can be generated and output. Specifically, a pattern in which the body temperature change rate rises for a certain period of time (e.g., a pattern in which a rise of about 0.3° C. per 10 minutes or more continues) can be determined as a feature indicating rapid eating. However, the threshold and time measurement criteria may be personalized and adjusted by the machine learning model according to user-specific physiological characteristics.

Next, it includes the edge AI unit calculating a change rate ratio of the heart rate information and blood pressure information to generate a cardiovascular response index (S230).

In step (S230), the edge AI unit of the wearable device (210) calculates a change rate ratio of heart rate information and blood pressure information to generate a cardiovascular response index.

The cardiovascular response index is defined as the ratio of blood pressure change to heart rate change (ΔBP/ΔHR), and is an indicator that quantifies the cardiovascular system's response to meals. This index is calculated as follows:

$$Cardiovascular response index = Δ BP / Δ HR$$

where ΔBP represents the change in blood pressure and ΔHR represents the change in heart rate.

The cardiovascular response index is used as an important indicator for determining meal amount and whether overeating has occurred. In particular, when the cardiovascular response index is less than −0.3, it is determined as an overeating state and overeating state information is generated and output, and when it is less than −0.5, it can be determined as a severe overeating state.

Next, it includes the edge AI unit inputting the body temperature derivative information, body temperature integral information, and cardiovascular response index into a machine learning model to predict eating behavior, meal amount, and/or nutrient composition (S240).

In step (S240), the edge AI unit of the wearable device (210) inputs body temperature derivative information, body temperature integral information, and cardiovascular response index into a machine learning model to predict eating behavior, meal amount, and/or nutrient composition.

The machine learning model is configured with a hybrid structure of Long Short-Term Memory (LSTM) and Temporal Convolutional Network, and is configured to simultaneously analyze a standard deviation of body temperature derivative information, a coefficient of variation of cardiovascular response index, and a slope of body temperature integral information within a time window of 10 to 20 minutes.

The edge AI unit estimates calorie intake according to the magnitude of body temperature integral information, and classifies protein, carbohydrate, and fat ratios according to the pattern of body temperature derivative information. In particular, when the slope of body temperature derivative information is at a rate of at least 0.3° C. per 20 minutes and not more than 0.7° C. per 20 minutes and the duration is at least 15 minutes and not more than 25 minutes, it is classified as a high-protein diet and high-protein diet information is generated, and when the slope of body temperature derivative information is at a rate of at least 0.1° C. per 20 minutes and not more than 0.3° C. per 20 minutes and the duration is at least 10 minutes and less than 15 minutes, it is classified as a high-fat diet and high-fat diet information is generated.

Next, it includes the wearable device (210) providing the prediction result to a user through an output unit or transmitting the prediction result to a user device (200) or service server (100) (S250).

In step (S250), the wearable device (210) provides the prediction result to a user through an output unit or transmits the prediction result to a user device (200) or service server (100).

The prediction result includes eating behavior, meal amount, nutrient composition ratio, etc., and may be directly provided to the user through an output unit of the wearable device (210) or transmitted to the user device (200) or service server (100).

The wearable device (210) transmits eating behavior prediction results, meal amount estimation results, and nutrient composition ratio analysis results generated by the edge AI unit, together with body temperature information, heart rate information, and blood pressure information measured at regular intervals over a 24-hour cycle, to the service server (100), and may receive an updated edge AI unit with improved analysis accuracy from the service server (100) and automatically update it.

Meanwhile, the wearable device (210) may receive and install an edge AI unit from the service server (100) or user device (200). The received edge AI unit is configured to include a lightweight AI model optimized for hardware performance of the wearable device (210).

Such an edge AI unit can process and analyze biological signals in real time on the wearable device (210) and provide immediate feedback without an internet connection. The edge AI unit is implemented to be optimized for limited computing resources of the wearable device (210), and is optimized for hardware performance of the wearable device (210) through quantization and model compression techniques.

The method for analyzing eating behavior based on biological signals according to an embodiment of the present invention can provide personalized health management services by analyzing biological signals collected from the wearable device (210) to predict the user's eating behavior, meal amount, and nutrient composition. In particular, it is configured to analyze eating patterns using body temperature derivative information, body temperature integral information, and cardiovascular response index calculated from biological signals such as body temperature, heart rate, and blood pressure.

In the specification and drawings, preferred embodiments of the present invention have been disclosed, and although specific terms have been used, they are used only in a general sense to easily explain the technical content of the present invention and help understanding of the invention, and are not intended to limit the scope of the present invention. It is obvious to those of ordinary skill in the art to which the present invention pertains that other modifications based on the technical spirit of the present invention can be implemented in addition to the embodiments disclosed herein.

1. A method of operating a service server that receives biological signal data from at least one wearable device worn on or attached to a user's body and predicts eating behavior, the method comprising:
receiving, by the service server, body temperature information, heart rate information, and blood pressure information measured by a body temperature measurement unit, a heart rate measurement unit, and a blood pressure measurement unit provided in the wearable device from a user device;
generating body temperature derivative information representing a rate of body temperature change per unit time (dT/dt) from the body temperature information, and body temperature integral information representing a time-integrated cumulative value of body temperature elevation relative to a baseline body temperature;
calculating a change rate ratio of the heart rate information and the blood pressure information to generate a cardiovascular response index;
inputting the body temperature derivative information, the body temperature integral information, and the cardiovascular response index into a machine learning model to predict eating behavior, meal amount, and/or nutrient composition; and
transmitting the prediction result to the user device or linking the prediction result with medical treatment data of a medical institution.

2. The method of claim 1, further comprising:
generating, by the service server, an edge AI unit comprising part or all of the machine learning model; and
transmitting the generated edge AI unit to the user device or the wearable device,
wherein the edge AI unit is configured to process and analyze biological signals collected by the wearable device locally in real time and provide immediate feedback without an internet connection.

3. The method of claim 2, wherein the edge AI unit transmitted by the service server is configured to operate within limited computing resources of the wearable device, and is optimized for hardware performance of the wearable device through quantization and model compression techniques.

4. The method of claim 2, further comprising:
receiving, by the service server, analysis results processed through the edge AI unit from the wearable device, performing cloud-based secondary analysis based on the analysis results, generating an updated edge AI unit, and retransmitting the updated edge AI unit to the wearable device.

5. The method of claim 1, further comprising:
periodically updating, by the service server, the machine learning model by learning biometric information and actual eating behavior data collected from a plurality of users.

6. The method of claim 5, further comprising:
generating, by the service server, a user-specific profile of a specific user selected from the plurality of users to build a personalized prediction model; and
comparing actual eating behavior data and predicted values for an initial period of 5 to 10 days to calibrate model weights in a personalized manner.

7. The method of claim 6, further comprising:
generating, by the service server, a user-customized edge AI unit based on the user-specific profile and the calibrated model weights and transmitting the user-customized edge AI unit to the user device or the wearable device, thereby enabling personalized eating behavior prediction and analysis to be performed locally.

8. The method of claim 1, wherein the cardiovascular response index is defined as a ratio of blood pressure change to heart rate change (ΔBP/ΔHR), the method further comprising:
determining, by the service server, an overeating state when the cardiovascular response index is less than −0.3, generating overeating state information, and transmitting the overeating state information to the user device.

9. The method of claim 8, wherein the service server is configured to determine an overeating state when the cardiovascular response index is less than −0.5, generate overeating state information, and transmit the overeating state information to the user device.

10. The method of claim 1, further comprising:
estimating, by the service server, calorie intake according to a magnitude of the body temperature integral information, wherein the body temperature integral information correlates with a total amount of diet-induced thermogenesis, and classifying protein, carbohydrate, and fat ratios according to a pattern of the body temperature derivative information to generate nutrient composition ratio information; and
transmitting the estimated calorie amount and the nutrient composition ratio information to the user device.

11. The method of claim 10, further comprising:
generating, by the service server, an edge AI unit comprising an AI model for the calorie estimation and the nutrient composition ratio classification and transmitting the edge AI unit to the wearable device, thereby enabling the wearable device to analyze calorie intake and nutrient composition ratio in real time without an internet connection and provide immediate feedback.

12. The method of claim 10, wherein the generating the nutrient composition ratio information comprises:
classifying as a high-protein diet when a slope of the body temperature derivative information is at a rate of at least 0.3° C. per 20 minutes and not more than 0.7° C. per 20 minutes and a duration is at least 15 minutes and not more than 25 minutes, generating high-protein diet information, and transmitting the high-protein diet information to the user device, or classifying as a high-fat diet when the slope of the body temperature derivative information is at a rate of at least 0.1° C. per 20 minutes and not more than 0.3° C. per 20 minutes and the duration is at least 10 minutes and less than 15 minutes, generating high-fat diet information, and transmitting the high-fat diet information to the user device.

13. The method of claim 1, further comprising:
determining, by the service server, a measurement error when a correlation coefficient of measured values among a plurality of wearable devices is from 0.5 to less than 0.7, transmitting a self-calibration command to a corresponding device, and receiving a self-calibration result and utilizing the self-calibration result for device performance improvement.

14. The method of claim 13, further comprising:
generating, by the service server, a distributed edge AI unit for self-calibration and data synchronization among the plurality of wearable devices and transmitting the distributed edge AI unit to each wearable device, thereby enabling the plurality of wearable devices to autonomously calibrate and synchronize measured values by communicating with each other.

15. The method of claim 1, further comprising:
analyzing, by the service server, temporal change patterns of the body temperature information, the heart rate information, and the blood pressure information to distinguish breakfast, lunch, dinner, and snacks; and
generating eating habit analysis information based on the distinction result and transmitting the eating habit analysis information to the user device.

16. The method of claim 1, further comprising:
sharing, by the service server, anonymized biometric information and meal pattern data with a medical research institution with user consent; and
receiving an improved analysis algorithm from the medical research institution and updating the machine learning model.

17. The method of claim 16, further comprising:
generating, by the service server, an updated edge AI unit based on the improved analysis algorithm received from the medical research institution and transmitting the updated edge AI unit to the wearable device, thereby enabling analysis reflecting latest medical research results to be performed locally.

18. A service server that communicates with at least one wearable device worn on or attached to a user's body and/or a user device connected to the wearable device to analyze biological signals and predict eating behavior, the service server comprising:
a communication unit configured to receive body temperature information, heart rate information, and blood pressure information from the wearable device;
a data processing unit configured to generate body temperature derivative information representing a rate of body temperature change per unit time (dT/dt) from the body temperature information, and generate body temperature integral information representing a time-integrated cumulative value of body temperature elevation relative to a baseline body temperature, and generate a cardiovascular response index from the heart rate information and the blood pressure information;
an AI analysis unit configured to receive the body temperature derivative information, the body temperature integral information, and the cardiovascular response index as inputs and predict eating behavior, calorie intake, and nutrient composition ratio;
a database configured to store data from a plurality of users and maintain user-specific customized models; and
a result providing unit configured to transmit prediction results to the user device or link the prediction results with a medical institution server.

19. The service server of claim 18, further comprising an edge AI deployment unit configured to generate an edge AI unit comprising part or all of the AI analysis unit and transmit the edge AI unit to the wearable device or the user device,
wherein the edge AI unit is optimized for hardware performance of the wearable device or the user device and is configured to perform real-time local processing and analysis of biological signals.

20. A method in which a wearable device worn on or attached to a user's body collects biometric information and an edge AI unit included in the wearable device analyzes the collected biometric information to predict eating behavior, the method comprising:
collecting, by the wearable device, body temperature information, heart rate information, and blood pressure information through a body temperature measurement unit, a heart rate measurement unit, and a blood pressure measurement unit, respectively;
generating, by the edge AI unit of the wearable device, body temperature derivative information representing a rate of body temperature change per unit time (dT/dt) from the body temperature information, and body temperature integral information representing a time-integrated cumulative value of body temperature elevation relative to a baseline body temperature;
calculating, by the edge AI unit, a change rate ratio of the heart rate information and the blood pressure information to generate a cardiovascular response index;
inputting, by the edge AI unit, the body temperature derivative information, the body temperature integral information, and the cardiovascular response index into a machine learning model to predict eating behavior, meal amount, and/or nutrient composition; and
providing, by the wearable device, the prediction result to a user through an output unit or transmitting the prediction result to a user device or a service server.

21. The method of claim 20, wherein the wearable device is a finger-worn device, a wrist-worn device, an ear-worn device, a chest-attached device, or a glasses-type device, the method further comprising:
performing, by the wearable device, self-calibration when a correlation coefficient of measured values among the devices is from 0.5 to less than 0.7.

22. The method of claim 20, wherein the body temperature integral information is a cumulative value of body temperature rise due to diet-induced thermogenesis (DIT), the method comprising:
continuously measuring, by the wearable device, body temperature of the user;
calculating, by the edge AI unit of the wearable device, a body temperature change rate over time from the measured body temperature data; and
determining, by the edge AI unit of the wearable device, rapid food intake when the calculated body temperature change rate exceeds a predetermined threshold.

23. The method of claim 22, further comprising:
generating and outputting, by the edge AI unit of the wearable device, rapid eating state information when a section in which the body temperature change rate is at a rate of 0.3° C. per 10 minutes or more and continues for 10 minutes or more is confirmed within a measurement period of 30 to 40 minutes.

24. The method of claim 20, wherein the cardiovascular response index is defined as a ratio of blood pressure change to heart rate change (ΔBP/ΔHR), the method further comprising:
determining, by the edge AI unit of the wearable device, an overeating state when the cardiovascular response index is less than −0.3, generating overeating state information, and outputting the overeating state information.

25. The method of claim 24, wherein the edge AI unit of the wearable device is configured to determine an overeating state when the cardiovascular response index is less than −0.5, generate overeating state information, and output the overeating state information.

26. The method of claim 20, further comprising:
a preprocessing step in which the edge AI unit of the wearable device distinguishes exercise-induced body temperature rise from meal-induced body temperature rise using exercise information collected through an accelerometer provided in the wearable device.

27. The method of claim 20, wherein the machine learning model is configured with a hybrid structure of Long Short-Term Memory (LSTM) and Temporal Convolutional Network (TCN), and is configured to simultaneously analyze a standard deviation of the body temperature derivative information, a coefficient of variation of the cardiovascular response index, and a slope of the body temperature integral information within a time window of 10 to 20 minutes.

28. The method of claim 20, further comprising:
estimating, by the edge AI unit of the wearable device, calorie intake according to a magnitude of the body temperature integral information, wherein the body temperature integral information correlates with a total amount of diet-induced thermogenesis, and classifying protein, carbohydrate, and fat ratios according to a pattern of the body temperature derivative information.

29. The method of claim 28, wherein the edge AI unit of the wearable device is configured to classify as a high-protein diet when a slope of the body temperature derivative information is at a rate of at least 0.3° C. per 20 minutes and not more than 0.7° C. per 20 minutes and a duration is at least 15 minutes and not more than 25 minutes, generate high-protein diet information, and transmit the high-protein diet information to the user device or the service server, and classify as a high-fat diet when the slope of the body temperature derivative information is at a rate of at least 0.1° C. per 20 minutes and not more than 0.3° C. per 20 minutes and the duration is at least 10 minutes and less than 15 minutes, generate high-fat diet information, and transmit the high-fat diet information to the user device or the service server.

30. The method of claim 20, further comprising:
receiving and installing, by the wearable device, an edge AI unit from a service server or a user device,
wherein the received edge AI unit is configured to include an AI model optimized for hardware performance of the wearable device.

31. The method of claim 20, further comprising:
transmitting, by the wearable device, eating behavior prediction results, meal amount estimation results, and nutrient composition ratio analysis results generated by the edge AI unit, together with body temperature information, heart rate information, and blood pressure information measured at regular intervals over a 24-hour cycle, to the service server, and receiving an updated edge AI unit with higher prediction accuracy than a previous version from the service server and automatically updating the edge AI unit.
